(19) United States
(12) Patent Application Publication
(10) Pub. No.: US 2024/0138917 A1
(43) Pub. Date: May 2, 2024

(54) APPARATUS FOR SIMULATING MEDICAL PROCEDURE PERFORMING CERVICAL ARTIFICIAL DISC SURGERY AND METHOD THEREOF

(71) Applicant: HealthHub Co.,Ltd., Seoul (KR)

(72) Inventors: Byung Hoon LEE, SEOUL (KR); Byung Il LEE, Seoul (KR); Shi Sub BYON, Seoul (KR)

(73) Assignee: HealthHub Co.,Ltd., Seoul (KR)

(21) Appl. No.: 18/258,983

(22) PCT Filed: Oct. 18, 2021
(86) PCT No.: PCT/KR21/14447

§ 371 Date: Jan. 2, 2024

(30) Foreign Application Priority Data
Dec. 23, 2020 (KR) 10-2020-0181593

(51) Int. Cl. A61B 34/10; A61F 2/46

(52) U.S. Cl. CPC A61B 34/10; A61F 2/4611; A61B 2034/104; A61B 2034/105; A61B 2034/108; A61F 2002/4633

(57) ABSTRACT

The present invention relates to an apparatus for simulating medical procedure performing cervical artificial disc surgery and a method thereof, for simulating the movement of the cervical artificial disc, when estimating a plurality of landmarks for a cervical artificial disc to be operated, generating an available space into which the cervical artificial disc is to be inserted by using the estimated landmarks, designing the cervical artificial disc model in the generated available space, and applying the designed cervical artificial disc model to a corresponding disc, prior to performing medical procedure of the cervical artificial disc surgery.

### TECHNICAL FIELD

The present invention relates to an apparatus for simulating medical procedure performing cervical artificial disc surgery and a method thereof, and more particularly, to an apparatus and method for simulating the movement of the cervical artificial disc, when estimating a plurality of landmarks for a cervical artificial disc to be operated, generating an available space into which the cervical artificial disc is to be inserted by using the estimated landmarks, designing the cervical artificial disc model in the generated available space, and applying the designed cervical artificial disc model to a corresponding disc, prior to performing medical procedure of the cervical artificial disc surgery.

### BACKGROUND

The human spine is a body organ forming a center of the human body and is composed of 7 cervical vertebrae (neck bones), 12 thoracic vertebrae (back bones), 5 lumbar vertebrae (lower back bones), 5 sacral vertebrae (pelvic bones), and 4 coccygeal vertebrae (tail bones).

The spine is positioned in the longitudinal direction at the center of the back of the body, supports the head on upper side, is connected downward to a pelvis, and transmits body weight to lower limb. In addition, a fiber cartilage intervertebral disc (i.e., a disc) is formed between vertebrae, so that strong ligament and muscle are combined from skull to pelvic bone, thereby supporting body and maintaining balance.

Poor posture, excessive physical activity, degenerative diseases, and the like cause a disease in a spine, and physical therapy is performed for a mild disease, but separate spinal fixation devices (for example, an implant such as a cervical artificial disc) should be used for treating the spine for a severe disease.

However, since a system for preoperatively simulating the movement of the cervical artificial disc inserted into the surgical site through a separate software device is not constructed, there is a problem in that the possibility of success of surgery is reduced or the satisfaction of users is degraded due to unfavorable postoperative prognosis, in the conventional surgical methods of a cervical artificial disc replacement surgery.

Accordingly, the present invention provides a method for estimating a plurality of landmarks for a cervical artificial disc to be operated, generating an available space into which the cervical artificial disc is to be inserted by using the estimated landmarks, and simulating the movement of the cervical artificial disc designed to be optimized in the generated available space, prior to performing a medical procedure for the cervical artificial disc.

Hereinafter, prior arts existing in the technical field of the present invention are briefly described, and the technical aspects that distinguish the present invention from the prior arts are described.

First, Korean Patent No. 0921722 (Oct. 15, 2009) relates to an apparatus and a method for evaluating an artificial cervical disc, and more particularly, to an apparatus and a method being configured to calculate a rotational axis coordinate of an artificial cervical disc captured and digitized by an X-ray imaging apparatus, and compare the rotational axis coordinate with a rotational axis of a cervical disc having a normal distribution, thereby capable of evaluating whether the operation of the cervical artificial disc is normal or not, and thus allow for the evaluation of the normality for a cervical movement influenced by cervical artificial disc and enable the design of the artificial disc that provides the most suitable mobility.

That is, the prior art (KR 0921722 B1) discloses an apparatus and a method configured to determine whether a rotation axis of a patient after surgery of the artificial cervical disc is within a normal distribution range, thereby reflecting it to the design of the artificial cervical disc, evaluate whether the devices can normally implement rotation in various cases including an artificial cervical disc, display the rotational axis distribution of the normal cervical vertebrae, and thereby capable of determining whether the rotation axis of the patient before surgery is within a normal distribution range.

On the other hand, since the present invention discloses estimating a plurality of landmarks for a cervical artificial disc to be operated, modeling an available space into which the cervical artificial disc is to be inserted by using the estimated landmarks, and simulating the movement of the cervical artificial disc designed according to the modeled available space, there are clear technical differences between the prior art (KR 0921722 B1) and the present invention in terms of their technical configurations.

In addition, Korean Patent No. 1347916 (Dec. 27, 2013) relates to artificial disc templating and placement in a spine, which enables a surgeon to select an optimal size of an intervertebral disc, and to place the intervertebral disc in an accurate location between the vertebral spaces to optimize athletic functionality.

In other words, the prior art (KR 1347916 B1) can be used by surgeons performing surgery for the actual replacement of a prosthetic disc, by one of a method of manually performing artificial disc fixation according to preference of a surgeon, a pedicle base circumference outline method combined with intraoperative fluoroscopy during intervertebral disc surgery, a method of automated prosthetic disc placement utilizing a two-ring aligning apparatus and drill guide method, and a method utilizing other commercially available registration software (such as computer tomography/fluoroscopy).

However, according to the present invention, it is possible to design a cervical artificial disc customized for each user by modeling the available space into which the cervical artificial disc to be operated is to be inserted, generating a cervical artificial disc model most suitable for the user, simulating the movement of the generated cervical artificial disc model, and adjusting the size or shape of the cervical artificial disc model on the basis of the simulated result, thereby there are significant structural differences between the prior art (KR 1347916 B1) and the present invention.

### BRIEF SUMMARY OF THE EMBODIMENTS

The present invention has been created to solve the aforementioned problems, and it is an objective of the present invention to provide an apparatus and a method thereof for conveniently simulating, through a user interface, a process of designing and simulating a cervical artificial disc in a customized manner for each user before performing an operation when the cervical disc replacement is performed due to an abnormality in the neck bone disc.

It is another objective of the present invention to provide an apparatus and a method thereof for intuitively and easily, through a user interface, generating an available space (i.e., A-space) into which a cervical artificial disc defined by a plurality of landmarks estimated from medical images for each user who is scheduled for surgery is to be inserted, generating a cervical artificial disc model optimized for the available space, and simulating movement at a time when the generated cervical artificial disc is applied to a surgical site.

It is another objective of the present invention to provide an apparatus and a method thereof for simulating movement after a cervical artificial disc is implanted, and precisely manufacturing a cervical artificial disc through a 3D printer with reference to the simulation result, in case of implanting a cervical artificial disc designed to be optimized for a state of a user to be operated in a surgical site.

It is another objective of the present invention to provide an apparatus and a method thereof for increasing the possibility of surgery success of cervical artificial disc replacement and improving the satisfaction of a user by allowing the cervical artificial disc to be manufactured in a customized manner according to the state of each user through the simulation.

It is characterized in that an apparatus for simulating medical procedure performing cervical artificial disc surgery in accordance with an embodiment of the present invention, comprises: an available space display part configured to display an available space defined by a plurality of landmarks estimated from a medical image of a user; a cervical artificial disc model display part configured to display the cervical artificial disc modeled to fit the available space; and a simulation response display part configured to display a movement of bending or unfolding a neck when the cervical artificial disc is applied to a surgical site.

In addition, it is characterized in that the apparatus further comprises: a medical image display part configured to display the medical image of the user; a surgical site area display part configured to display the surgical site area according to the designation of the surgical site area of an expert who has examined the medical image of the user; and a landmark display part configured to display the plurality of landmarks estimated from the surgical site area.

In addition, it is characterized in that the apparatus further comprises: an A-space inspection part configured to inspect an A-space by comparing the A-space, which is an available space defined by the plurality of landmarks estimated from the medical image of the user, with a disc space of the surgery site; a cervical artificial disc coupling part configured to apply a cervical artificial disc model modeled according to the A-space to the surgical site of the user to be operated; and a simulation condition setting part configured to set a condition for simulating the movement of the neck when the cervical artificial disc model is applied to the surgical site of the user to be operated, wherein the simulation result according to the set simulation condition is displayed through the simulation response display part.

Wherein, it is characterized in that the available space is the A-space defined by the plurality of landmarks, and the A-space is that a space between two unit cervical vertebrae to which the cervical artificial disc is implanted is modeled using the plurality of landmarks.

Wherein, it is characterized in that the plurality of landmarks are configured to be set: at a center and left and right sides of a front outer region of a lower end of an upper end unit cervical vertebra to a direction of a skull, a cranial anterior center, a cranial anterior right, and a cranial anterior left, respectively; at the center of the lower end of the upper end unit cervical vertebra to the direction of the skull, a cranial apex; at a center and left and right sides of a rear outer region of the lower end of the upper end unit cervical vertebra to the direction of the skull, a cranial posterior center, a cranial posterior right, and a cranial posterior left, respectively; at a center and left and right sides of a front outer region of an upper end of a lower end unit caudal vertebra to a direction of a spine, a caudal anterior center, a caudal anterior near right, a caudal anterior far right, a caudal anterior near left, and a caudal anterior far left, respectively; and at a center and left and right sides of a rear outer region of the upper end of the lower end unit caudal vertebra to the direction of the spine, a caudal posterior center, a caudal posterior near right, a caudal posterior far right, a caudal posterior near left, and a caudal posterior far left, respectively.

Moreover, it is characterized in that a method for simulating medical procedure performing cervical artificial disc surgery in an apparatus thereof in accordance with an embodiment of the present invention, is configured to comprises: displaying an available space defined by a plurality of landmarks estimated from a medical image of a user; displaying a cervical artificial disc modeled to fit the available space; and displaying a movement of bending or unfolding a neck when the cervical artificial disc is applied to a surgical site.

In addition, it is characterized in that the method further comprises displaying a medical image of a user; displaying the surgical site area according to a designation of the surgical site area by an expert who has examined the medical image of the user; and displaying the plurality of landmarks estimated from the surgical site area.

In addition, it is characterized in that the method further comprises: inspecting an A-space by comparing the A-space, which is an available space defined by the plurality of landmarks estimated from the medical image of the user, with a disc space of the surgery site; applying a cervical artificial disc model modeled according to the A-space to a surgical site of the user to be operated; and setting a condition for simulating the movement of the neck when the cervical artificial disc model is applied to a surgical site of the user to be operated, wherein the simulation apparatus is configured to display the simulation result according to the set simulation condition on a screen.

As described above, an apparatus for simulating medical procedure performing cervical artificial disc surgery and method thereof in accordance with the present invention, intuitively and conveniently process, through a user interface, generating an available space into which a cervical artificial disc of a user to be operated is inserted by using a plurality of landmarks and simulating movement with respect to a cervical artificial disc model designed to be optimized in the available space, so that the cervical artificial disc can be manufactured in a customized manner with reference to the simulation result, and it is promising that the possibility of success of surgery of cervical artificial disc replacement is increased and the satisfaction of a user related to surgery prognosis is improved.

### BRIEF DESCRIPTION OF THE DRAWINGS

FIG. 1 is a conceptual view schematically illustrating a usage environment of a simulation apparatus for medical procedure performing cervical artificial disc surgery according to an embodiment of the present disclosure.

FIG. 2 is a diagram illustrating a simulation process for medical procedure performing cervical artificial disc surgery according to an embodiment of the present disclosure.

FIG. 3 is a view illustrating a plurality of landmarks set in vertical cervical vertebrae in which a cervical artificial disc is implanted, and an A-space generated by the plurality of landmarks according to an embodiment of the present disclosure.

FIG. 4 is a view for explaining, in more detail, positions of landmarks applied to the present disclosure.

FIG. 5 is a block diagram illustrating, in more detail, a configuration of a simulation apparatus for medical procedure performing cervical artificial disc surgery according to an embodiment of the present disclosure.

FIG. 6 is a view illustrating, in more detail, a configuration of a simulation result display part according to an embodiment of the present disclosure.

FIGS. 7A to 7J are views showing examples of screen states displayed through a simulation result display part according to an embodiment of the present disclosure.

FIG. 8 is a flowchart illustrating an operational process of a simulation method for medical procedure performing cervical artificial disc surgery according to an embodiment of the present disclosure.

### DETAILED DESCRIPTION OF THE EXEMPLARY EMBODIMENTS

Hereinafter, exemplary embodiments of an apparatus for simulating medical procedure performing cervical artificial disc surgery and method thereof according to the present invention are described in detail with reference to the accompanying drawings. The identical reference numerals indicated on each of the drawings denote the same components. In addition, specific structural or functional descriptions for embodiments of the present invention are only exemplified for the purpose of describing embodiments according to the present invention, and unless otherwise defined, all terms used herein, including technical or scientific terms, have the same meanings as commonly understood by one of ordinary skill in the art to which the present disclosure pertains. Commonly used terms, such as those defined in a dictionary should be interpreted to have a meaning that is consistent with their meaning in the context of the relevant art, and it is desirable that the terms are not to be interpreted in an idealized or overly formal sense unless explicitly defined herein in the specification.

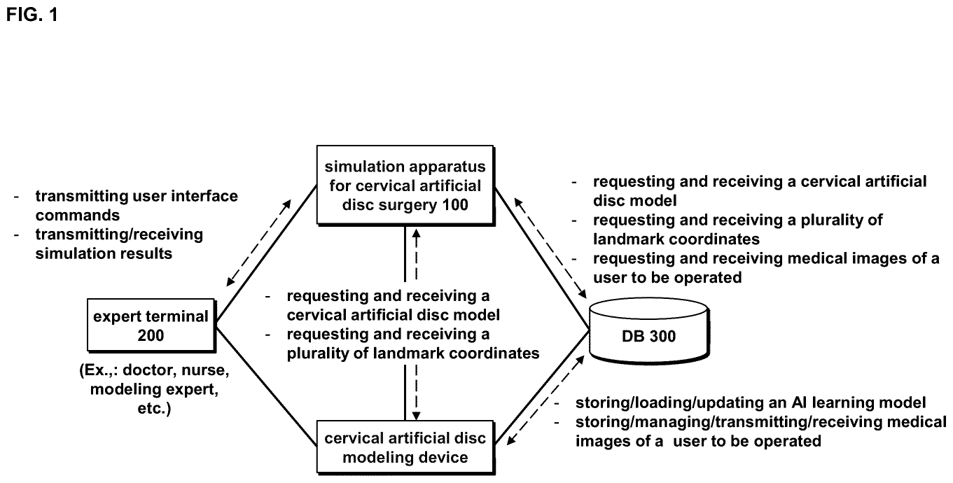

FIG. 1 is a conceptual view schematically illustrating a usage environment of a simulation apparatus for medical procedure performing cervical artificial disc surgery according to an embodiment of the present disclosure.

As shown in FIG. 1, the present invention is illustrated with an apparatus for simulating medical procedure performing cervical artificial disc surgery 100 (hereinafter, referred to as a simulation apparatus), a modeling apparatus for a cervical artificial disc, an expert terminal 200, a database 300, and the like.

The simulation apparatus 100 is a simulation tool for intuitively and conveniently performing, through a user interface, the entire process of designing and simulating a cervical artificial disc according to the condition of a user's disease or cervical vertebrae by an expert, such as a doctor, before performing cervical artificial disc replacement or the like due to abnormality in a neck bone (that is, cervical vertebrae).

For example, the simulation apparatus 100 is configured to perform, through a user interface, loading medical image of a surgery scheduled user, three-dimensional modeling of a specific cervical vertebrae designated by a doctor, generating an A-space, which is an available space into which the cervical artificial disc is to be inserted, by using a plurality of landmarks estimated from medical image for a part of a cervical vertebrae for a user, modeling a cervical artificial disc modeling optimized for the A-space, and simulating a movement when the modeled cervical artificial disc is applied.

In this case, a plurality of landmarks are set (for example, 7 landmarks are set at lower end of a upper end unit cranial vertebra to a skull direction and 10 landmarks are set at upper end of lower end unit cervical vertebrae to a spine direction) at lower end of upper end unit cranial vertebrae to a skull direction and at upper end of lower end unit caudal vertebrae, respectively, in a specific joint (two unit cervical vertebrae and a disc there between) of the medical image).

Wherein, the A-space is an available space defined by the plurality of landmarks, and models a space between two unit cervical vertebrae to which the cervical artificial disc is implanted.

In addition, the simulation apparatus 100 is configured to adjust a size or shape of a cervical artificial disc by referring to a simulation result obtained when the cervical artificial disc designed to be optimized for a state of a user is applied to a surgical site, thereby designing a customized cervical artificial disc for each user.

That is, through the simulation, the cervical artificial disc is manufactured in a customized manner according to the state of each user, thereby increasing the success probability of surgery of the cervical artificial disc replacement and improving the satisfaction of the user.

In addition, the simulation apparatus 100 is configured to request a user medical image to be operated, a plurality of landmark coordinates estimated from a medical image of the user, an A-space, and a cervical artificial disc model, and receive the same, and perform a simulation based on the received user medical image, the plurality of landmark coordinates, the A-space, the cervical artificial disc model, and the like.

In addition, the simulation apparatus 100 is configured to request a plurality of landmark coordinates estimated from medical image of a user, an A-space generated based on the plurality of landmarks, and a cervical artificial disc model, directly to a cervical artificial disc modeling apparatus, and receive the same.

Meanwhile, the simulation apparatus 100 is preferably manufactured and used as an independent tool. However, the present disclosure does not limit the simulation apparatus thereto, and the simulation apparatus 100 may be manufactured in the form of an application program and installed and used in the expert terminal 200.

The cervical artificial disc modeling apparatus is configured to estimate a plurality of landmarks from a medical image of a user, generate an available space (A-space) defined by the estimated plurality of landmarks, and models a cervical artificial disc model optimized for the generated A-space.

In addition, the cervical artificial disc modeling apparatus is configured to locally store and manage a plurality of landmarks estimated from a user medical image, an A-space generated using the plurality of landmarks, and a cervical artificial disc model optimized for the A-space, or store and manage the same through the database 300.

The expert terminal 200 is a wired/wireless communication terminal such as a smartphone, a tablet, a PC, etc. used by a medical team, an engineer that manufactures a disc, or the like.

In addition, the expert terminal 200 is configured to interact with the simulation apparatus 100 through operations of an expert, transmit various control commands related to disc modeling, simulation, or the like, and process the procedures of medical image loading, landmark estimation, A-space generation, cervical artificial disc modeling, movement simulation, or the like performed in the simulation apparatus 100 through a user interface.

The database 300 is configured to perform storing and update managing of various operation programs for supporting user medical image loading, in which the user medical image is used in the simulation apparatus 100, landmark estimation, A-space generation, cervical artificial disc modeling, movement simulation, or the like.

In addition, the database 300 is configured to store and manage member information (e.g., an ID, a password, contact information, etc.) of a user to be operated and a medical image obtained by photographing the cervical vertebrae, and store and manage a learning model for estimating the landmarks.

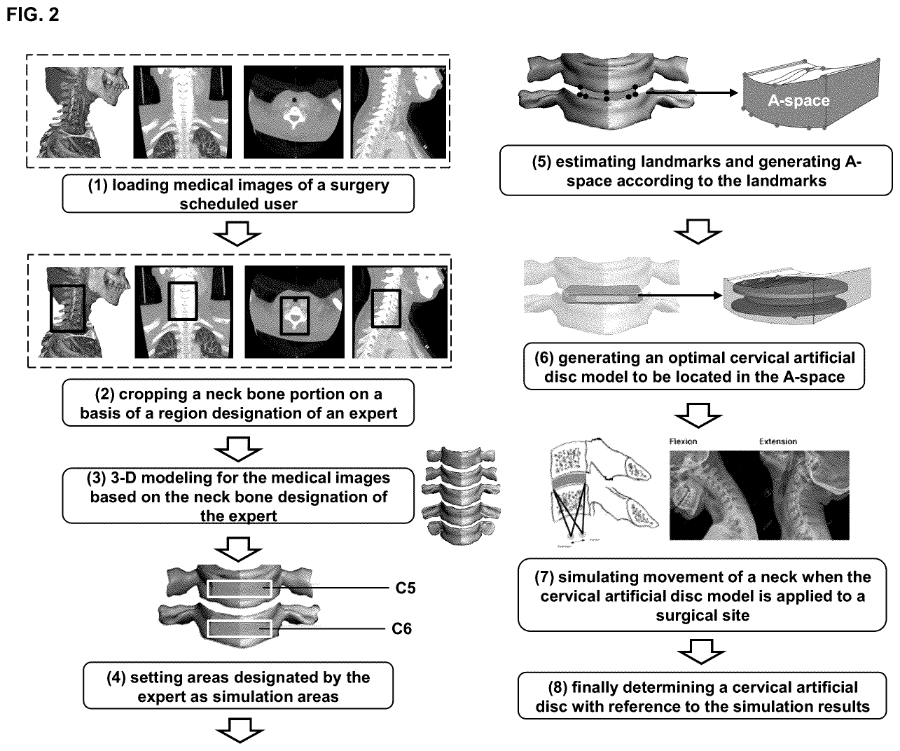

Meanwhile, hereinafter a simulation process for medical procedure of the cervical artificial disc surgery is described in detail with reference to FIG. 2.

FIG. 2 is a diagram illustrating a simulation process for medical procedure performing cervical artificial disc surgery according to an embodiment of the present disclosure.

As shown in FIG. 2, the simulation apparatus 100 is configured to load a medical image of a surgery scheduled user from the database 300 through operations of an expert and output the medical image on a screen (1).

In addition, the simulation apparatus 100 is configured to display by cropping a neck bone portion on a basis of a region designation of an expert who has examined the medical image of the user outputted on a screen (2), and then convert corresponding portion of a user medical image into a three-dimensional model and display the three-dimensional model on the screen (3).

In addition, when a part to proceed with surgery is designated by an expert who has examined a user medical image displayed as a 3D model, the simulation device 100 is configured to determine the corresponding parts (e.g., C5 and C6) as simulation areas and display the simulation areas on a screen (4).

Subsequently, the simulation apparatus 100 is configured to estimate a plurality of landmarks from medical image of a user, model an A-space, which is an available space defined by the plurality of estimated landmarks, and display the same on a screen (5).

In addition, the simulation apparatus 100 is configured to generate a cervical artificial disc to be located in an A-space and display the cervical artificial disc on a screen (6). For example, the simulation apparatus 100 is configured to model the cervical artificial disc model to be located in the A-space and display the model on a screen.

Thereafter, the simulation apparatus 100 is configured to simulate movement of a neck (for example, a flexion and extension exercise that bends the neck forward and stretches the neck), and display a simulation process on the movement on a screen (7), when the modeled cervical artificial disc is applied to the surgical site of a corresponding user.

In addition, the simulation apparatus 100 is configured to determine the final specification of the cervical artificial disc by adjusting the specification of the cervical artificial disc according to the movement of the corresponding user based on the simulation result information, and display information on the final specification on the screen to allow an expert or a user to identify the information (8).

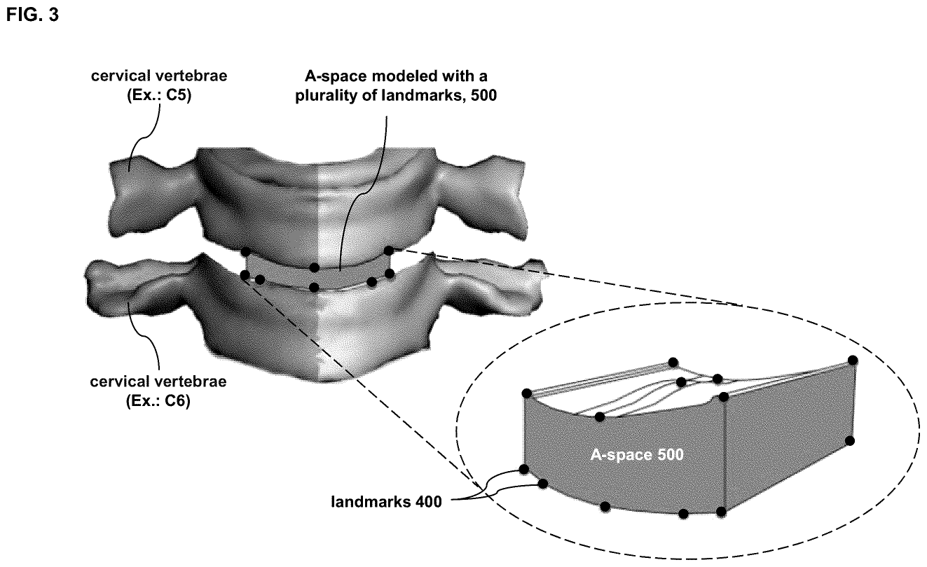

FIG. 3 is a view illustrating a plurality of landmarks set in vertical cervical vertebrae in which a cervical artificial disc is implanted, and an A-space generated by the plurality of landmarks according to an embodiment of the present disclosure.

As shown in FIG. 3, the A-space 500 is configured to be an available space defined by a plurality of landmarks 400, model a space between two unit cervical vertebrae to which the cervical artificial disc is to be coupled (e.g., between C5 cervical vertebrae and C6 cervical vertebrae), and be modeled in a left-right symmetric structure.

Wherein, the A-space 500 is modeled all differently depending on a cervical condition and a surgical site of a user to be operated.

Accordingly, when a cervical artificial disc suitable for a corresponding user is modeled by utilizing A-space 500 uniquely modeled for each user, it is possible to solve a problem of decreased surgical success probability that was occurred by surgery previously caused by using a cervical artificial disc designed according to predetermined specification.

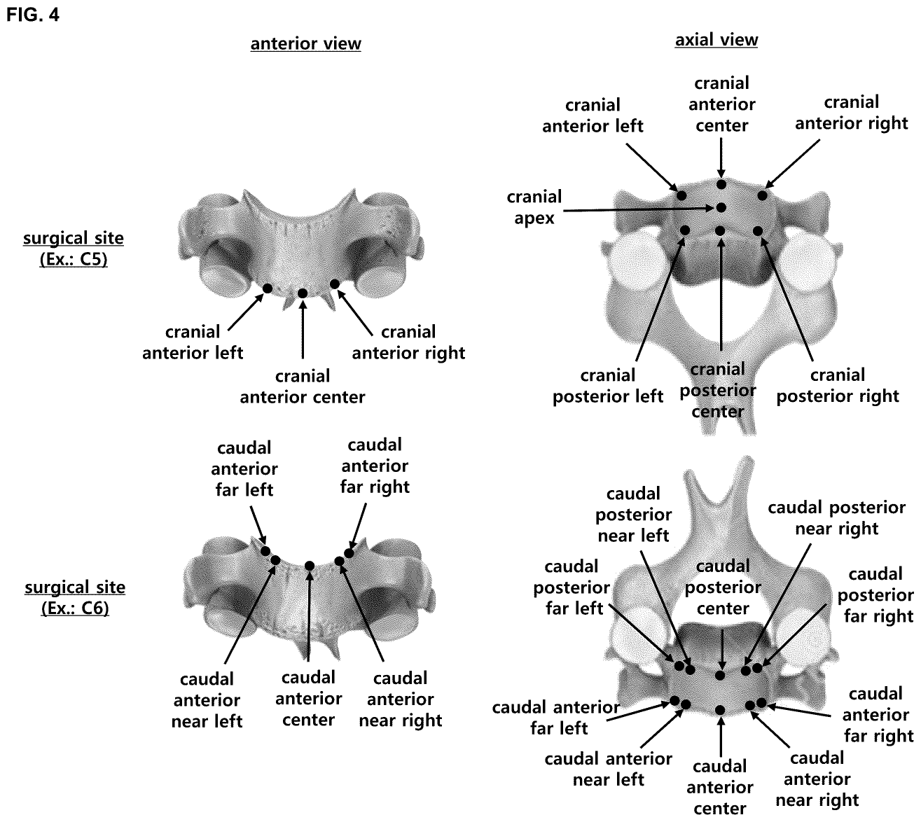

FIG. 4 is a view for explaining, in more detail, positions of landmarks applied to the present disclosure.

As shown in FIG. 4, a plurality of landmarks 400 are configured to be set with multiple landmarks for acquiring area sufficiently surrounding a unit cervical vertebra of a surgical site, and guiding the plurality of landmarks 400 to be implanted by aligning along a central line, and generate an A-space 500, which is an available space into which the cervical artificial disc is to be inserted, by the plurality of landmarks 400.

The landmarks 400 consists of 7 landmarks set on lower end tipper end unit cranial vertebrae to a skull direction and 10 landmarks set on upper end of lower unit caudal vertebrae to a spine direction, at a specific joint.

In other words, the landmarks 400 are configured to be set to comprise: on a center and left and right sides of a front outer region of the lower end of the upper end unit cervical vertebra to the direction of the skull, a cranial anterior center, a cranial anterior right, and a cranial anterior left, respectively; on a center of the lower end of the upper end unit cervical vertebra to the direction of the skull, a cranial apex; on a center and left and right sides of a rear outer region of the lower end of the upper end unit cervical vertebra to the direction of the skull, a cranial posterior center, a cranial posterior right, and a cranial posterior left, respectively.

In addition, the landmarks 400 are configured to be set to comprise: on a center and left and right sides of a front outer region of a upper end of a lower end unit caudal vertebra to a direction of a spine, a caudal anterior center, a caudal anterior near right, a caudal anterior far right, a caudal anterior near left, and a caudal anterior far left, respectively; and on a center and left and right sides of a rear outer region of the upper end of the lower end unit caudal vertebra to the direction of the spine, a caudal posterior center, a caudal posterior near right, a caudal posterior far right, a caudal posterior near left, and a caudal posterior far left, respectively.

Wherein, in the present invention, the landmarks 400 is set, as an example, with a total 17 landmarks, but the present invention does not limit thereto, and the number of landmarks may be increased or decreased as desired.

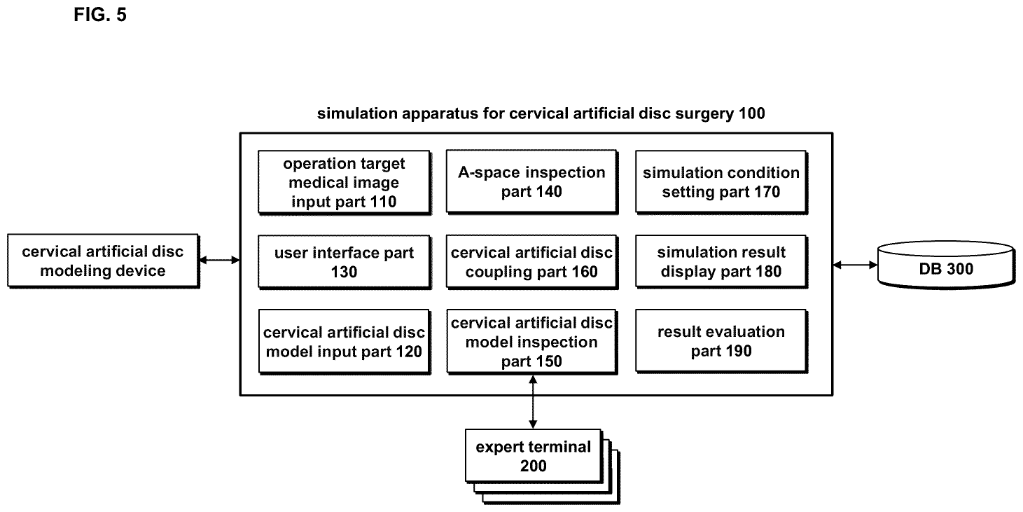

FIG. 5 is a block diagram illustrating, in more detail, a configuration of a simulation apparatus for medical procedure performing cervical artificial disc surgery according to an embodiment of the present disclosure.

As shown in FIG. 5, the simulation apparatus 100 comprises a medical image input part 110 for an operation target, a cervical artificial disc model input part 120, a user interface part 130, an A-space inspection part 140, a cervical artificial disc model inspection part 150, a cervical artificial disc coupling part 160, a simulation condition setting part 170, a simulation result display part 180, a result evaluation part 190.

Although not shown in the drawings, the simulation apparatus 100 may further comprise a power supply part for supplying operational power to each component, an input part for setting or inputting data for various functions, and an update management part for managing updates of various operational programs.

The medical image input part 110 for an operation target is configured to receive a medical image photographing a cervical part of a user pre-stored in the database 300, or a medical image obtained by photographing a cervical part of a user from a medical image capturing device such as a CT, an MIRI, etc.

The cervical artificial disc model input part 120 is configured to receive a cervical artificial disc model of a patient to be operated (that is, an operation scheduled user) modeled in a cervical artificial disc modeling apparatus.

The user interface part 130 is configured to perform functions of instructing and controlling an overall operation of the simulation apparatus 100 through a user interface according to an operation of an expert.

The A-space inspection part 140 is configured to inspect the A-space by receiving an A-space generated based on a plurality of landmarks estimated from the medical image of a surgery-scheduled user from a cervical artificial disc modeling apparatus, and comparing the A-space with corresponding disc space of the medical image of the surgery-scheduled user received from the medical image input part 110. The inspection by the comparison may be performed by identifying the coordinates of each of the landmarks in a state that a user (or an expert) inserts the A-space into the disc space.

The cervical artificial disc model inspection part 150 is configured to bring a cervical artificial disc model generated for medical image of a user to be operated from a cervical artificial disc modeling apparatus, and inspect whether the shape of a surface and edges matches upper and lower cervical vertebrae, compared to the medical image of the user to be operated. Wherein, a portion that the cervical artificial disc model does not match in comparison with the medical image of the user to be operated, may be automatically detected and displayed.

The cervical artificial disc coupling part 160 is configured to generate a combined image by combining the cervical artificial disc model identified in the cervical artificial disc model inspection part 150 with the medical image of a user to be operated. Wherein, a user may manually manipulate using a mouse to directly combine the cervical artificial disc model with the medical image of a user to be operated, but may automatically combine the cervical artificial disc model with the medical image of a user to be operated, with reference to the landmark coordinates.

The simulation condition setting part 170 is configured to be a part for setting a condition that an expert sets through a user interface part 130, that is, adds movement to the cervical artificial disc and upper and lower cervical vertebrae coupled through the cervical artificial disc coupling part 160. Wherein, the condition is configured to comprise a degree of matching when the cervical artificial disc model is combined with the medical image of an actual user, whether the cervical artificial disc is able to withstand the force applied in each direction of the cervical artificial disc, whether the cervical artificial disc comes into contact with other areas in case of applying movement of flexing a neck forward or extending the neck backward.

The simulation result display part 180 is configured to display a simulation result performed according to the simulation condition set by the simulation condition setting part 170 on a screen, and output the results simulating A-space, surface and edge of the cervical artificial disc, a combined image of the cervical artificial disc, and a response for an exercise condition on the screen.

Wherein, the simulation is an operation of identifying whether the cervical artificial disc model is matched with a medical image of an actual user through combining the cervical artificial disc model with a medical image of an actual user, whether the cervical artificial disc is able to withstand the force applied in each direction of the cervical artificial disc, and whether the cervical artificial disc comes into contact with other areas in case of applying movement of flexing a neck forward or extending the neck backward.

The result evaluation part 190 is configured to output error information with respect to the simulation result, in case of deviating a simulation result from a predetermined condition.

Furthermore, the result evaluation part 190 is also configured to identify a degree in which the simulation result is out of a predetermined condition, and guide a range of modifying the cervical artificial disc model. For example, the specification of the cervical artificial disc model is finally adjusted according to a neck movement of corresponding user based on the simulation result information.

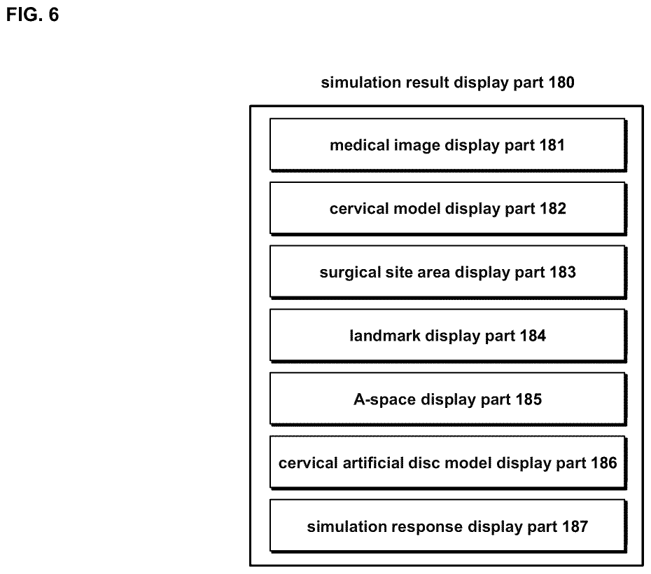

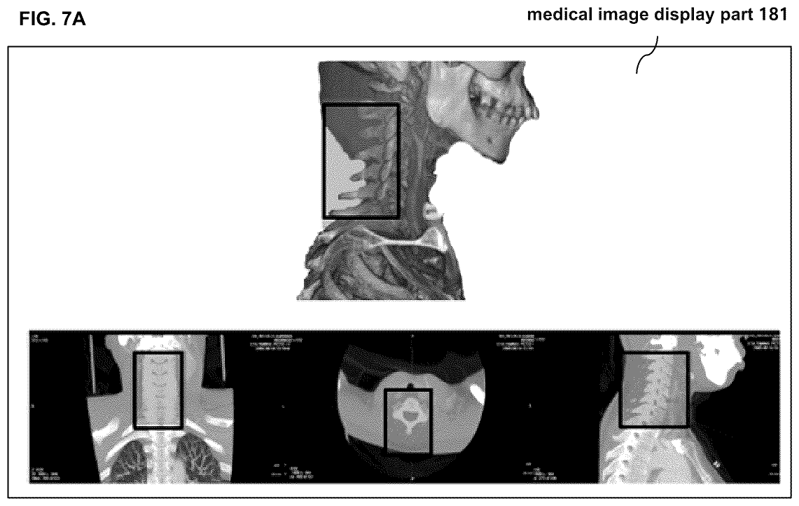

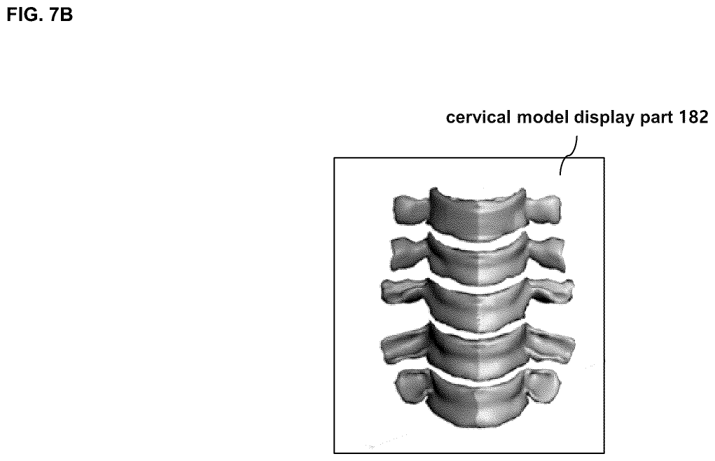

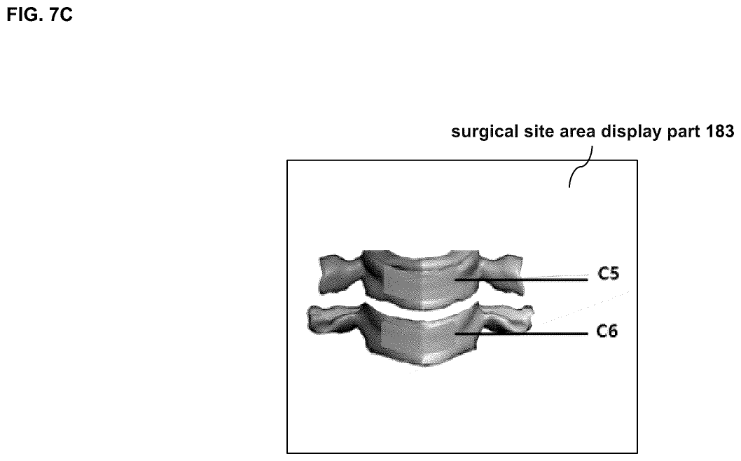

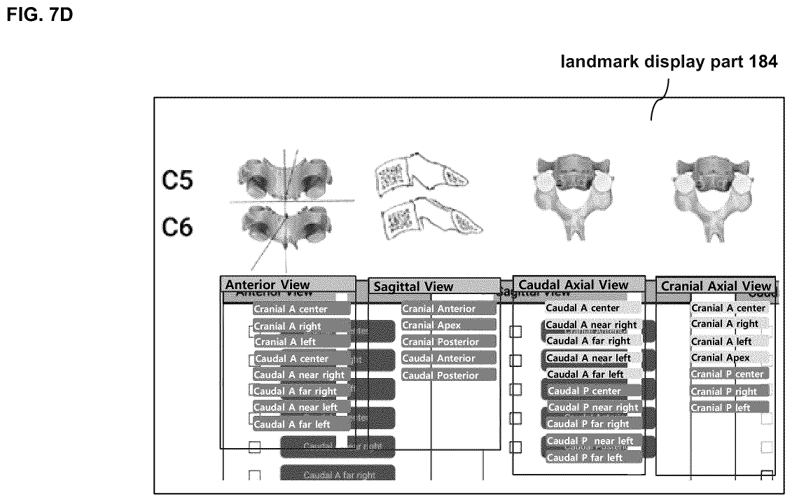

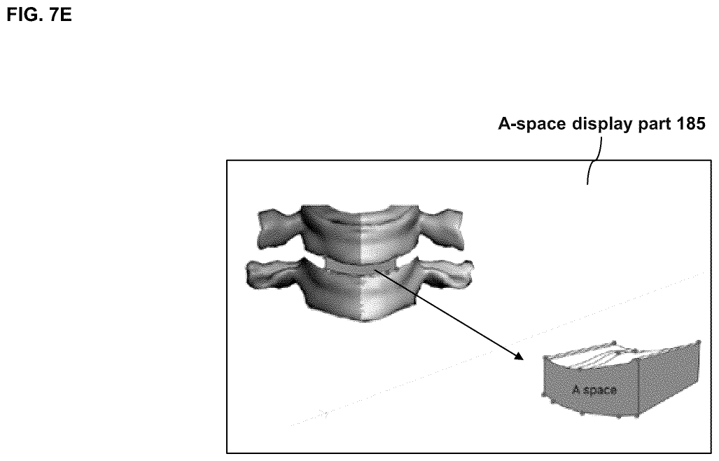

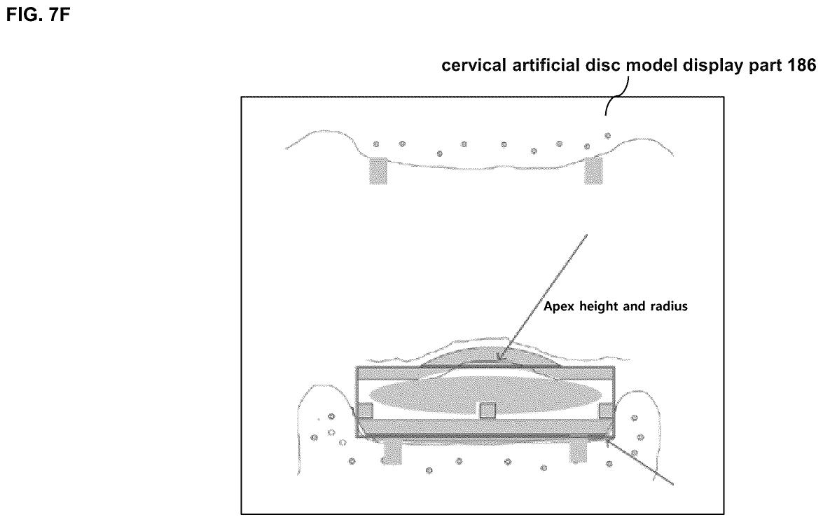

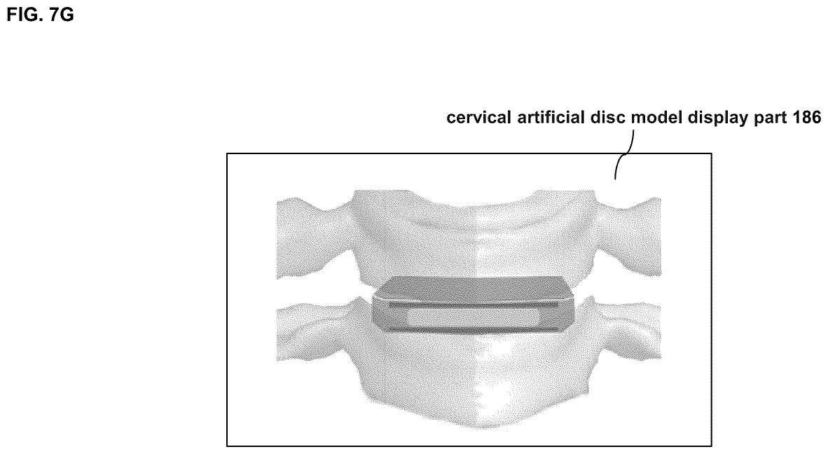

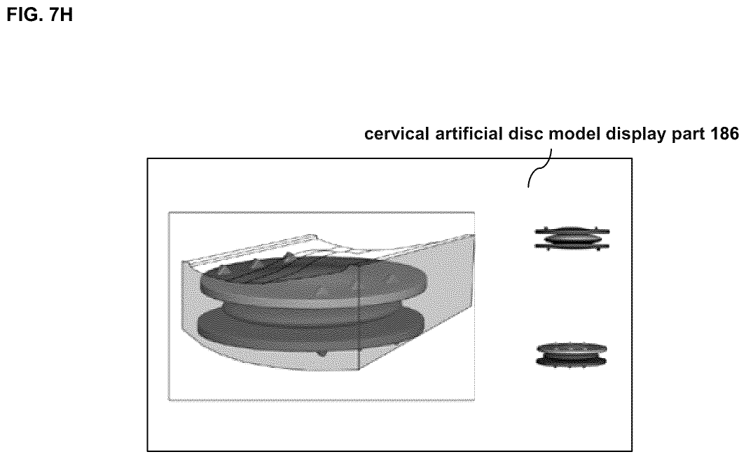

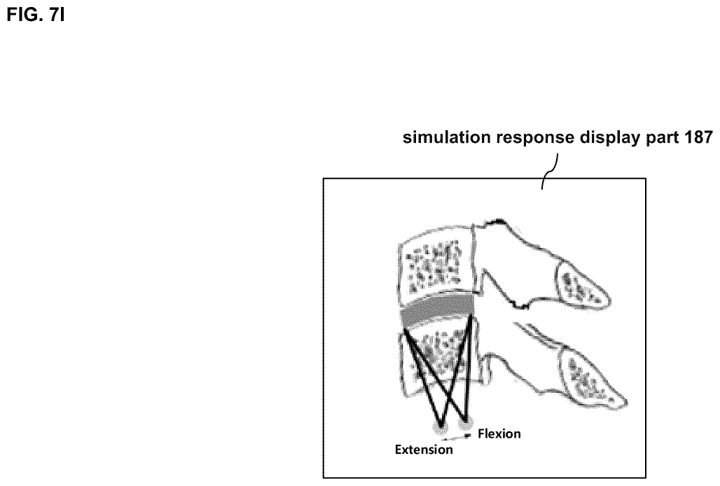

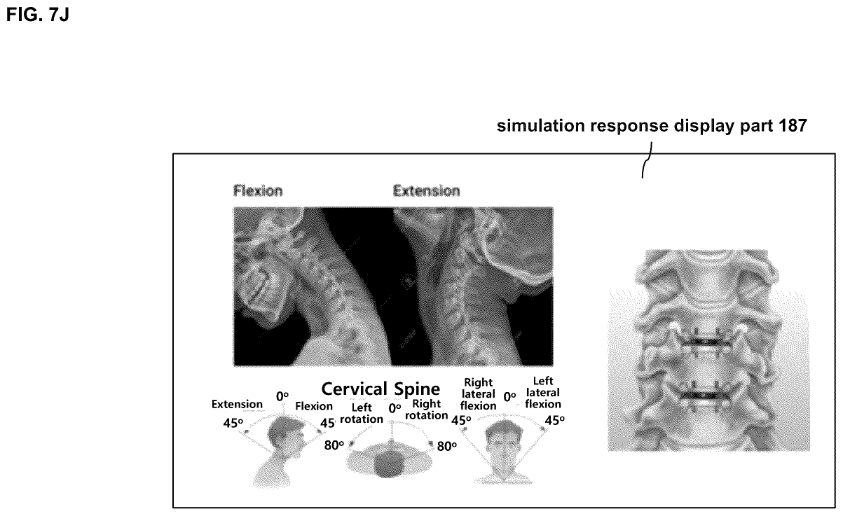

FIG. 6 is a view illustrating, in more detail, a configuration of a simulation result display part according to an embodiment of the present disclosure, and FIGS. 7A to 7J are views showing examples of screen states displayed through a simulation result display part according to an embodiment of the present disclosure.

As shown in FIG. 6, the simulation result display part 180 comprises a medical image display part 181, a cervical model display part 182, a surgical site area display part 183, a landmark display part 184, an A-space display part 185, a cervical artificial disc model display part 186, and a simulation response display part 187.

The medical image display part 181 is configured to display a medical image of a scheduled surgery user, which is stored in the database 300 or inputted to be photographed in real time from the medical image capturing device, and display the medical image of the user on a screen to identify the medical image of the user in a 3-dimensional manner from 6 perspectives, as shown in FIG. 7A.

When an expert (i.e., a doctor) designates a cervical part as an area of rectangular shape in a medical image of the user displayed on the medical image display part 181, the cervical model display part 182 is configured to crop a corresponding area to display the area as a three-dimensional model, convert the cervical part of the user into a three-dimensional model, and display the three-dimensional model on a screen, as shown in FIG. 7B.

When an expert designates a portion (that is, a portion requiring surgery) for inserting the cervical artificial disc in the medical image of the user displayed on the cervical model display part 182, the surgical site area display part 183 is configured to reduce only the corresponding portion and display the reduced portion as a three-dimensional model, and display a three-dimensional model of the medical image of the user with respect to the surgical site area on a screen, as shown in FIG. 7C.

For example, as a result of inspecting the 3D model displayed on the cervical model display part 182, an expert determines that the surgery is required between C5 and C6, and designates a corresponding part to a surgical site area through a mouse or a key input, the surgical site area display part 183 is configured to extract only the portion designated by the expert, reduce a simulation space and display the reduced simulation space on the screen.

The landmark display part 184 is configured to display a plurality of landmarks estimated from a specific surgical site area displayed through the surgical site area display part 183 on a screen, and the plurality of landmarks respectively estimated from upper and lower unit cervical vertebrae of a specific joint requiring surgery are displayed on a screen, as shown in FIG. 7D.

Wherein, the landmark display part 184 is configured to receive information on the landmarks directly from the database 300 or a cervical artificial disc modeling apparatus and display the received information on a screen, and otherwise it is also possible to apply a method where an expert directly sets landmarks on upper and lower unit cervical vertebrae of a specific joint having the expert directly three-dimensionally modeled on the medical image of the user, and displays the landmarks on the screen.

The A-space display part 185 is configured to display A-space, which is an available space defined by a plurality of landmarks estimated from a medical image of a user, and A-space defined as a plurality of landmarks is displayed on a screen, as shown in FIG. 7E.

The cervical artificial disc model display part 186 is configured to display a cervical artificial disc model modeled fit to an A-space displayed through the A-space display part 185, and as illustrated in FIGS. 7F to 7H, a cervical artificial disc model modeled by a height (e.g., an interval between upper vertex of a cranial (upper) plate and lower vertex of a caudal (lower) plate), a length (e.g., the width of the major axis), a width (e.g., the width of the minor axis), a shape (surface or edge), or a combination thereof (e.g., a specification of a cervical artificial disc model) is displayed on the screen.

The simulation response display part 187 is configured to display a movement of bending or unbending a neck when the cervical artificial disc model displayed through the cervical artificial disc model display part 186 is applied to a surgical site, and as shown in FIGS. 71 and 7J, a movement of coming the cervical artificial disc into contact with other areas or a movement of flexing a neck forward or extending the neck backward is displayed on a screen.

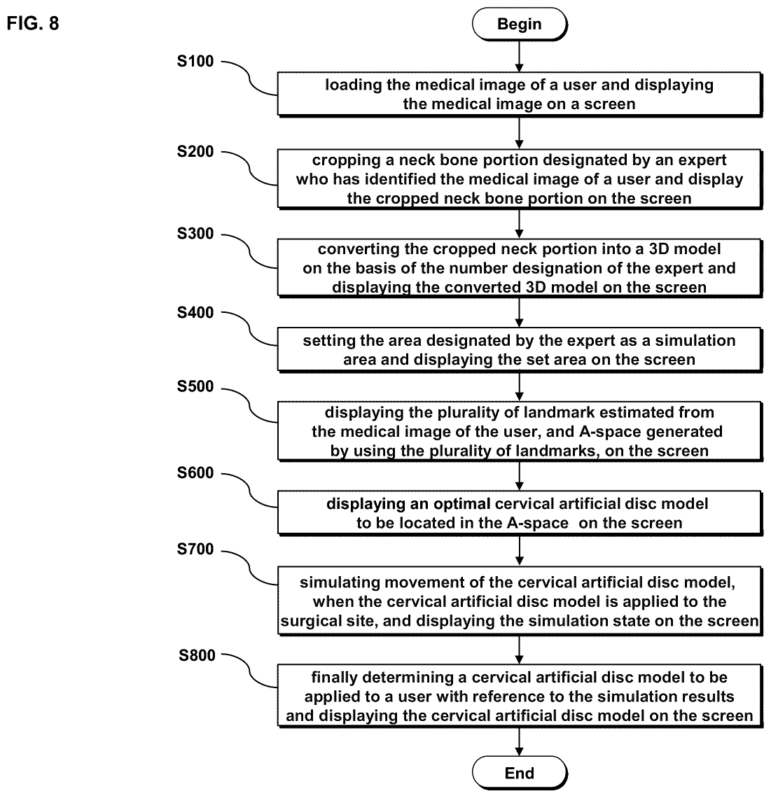

Hereinafter, an embodiment of a method for simulating medical procedure of cervical artificial disc surgery according to the present invention is described in detail with reference to FIG. 8. Wherein, each process according to the method of the present invention may be changed in order by a usage environment or a person skilled in the art.

FIG. 8 is a flowchart illustrating an operational process of a simulation method for medical procedure performing cervical artificial disc surgery according to an embodiment of the present disclosure.

As shown in FIG. 8, the simulation apparatus 100 is configured to display a medical image of a user stored in the database 300 on a screen according to an operation by an expert, or receive the medical image of the user photographed by a medical image photographing apparatus such as a CT or an MRI device and display the medical image on a screen S100. That is, the simulation apparatus 100 is configured to load the medical image of a user who needs cervical artificial disc replacement surgery, and output the medical image on the screen.

After loading the medical image of a user on the screen through S100, the simulation apparatus 100 is configured to perform cropping a neck bone portion on the basis of a region designation of an expert who has identified the medical image of a user and display the cropped neck bone portion on the screen, S200, convert the cropped neck portion into a 3D model on the basis of the number designation (for example, the numbers (C1 to C7) for cervical vertebrae 1 to 7) of the expert who has identified the cropped neck portion and display the converted 3D model on the screen, S300.

In addition, when the expert who has identified the cervical vertebrae of the surgery scheduled user displayed as the 3D model through S300 designates the area of the surgical site, the simulation apparatus 100 is configured to model the area of the surgical site designated by the expert and display the modeled area on the screen, S400. That is, the surgical site of a user who should perform the cervical artificial disc replacement surgery is set as a simulation area.

Subsequently, the simulation apparatus 100 configured to estimate a plurality of landmark coordinates from the medical image of a user with respect to the surgical site displayed on the screen through S400 and display the plurality of landmark coordinates on the screen, and generate an A-space, which is an available space defined by the plurality of estimated landmarks and display the generated A-space on the screen, S500. That is, the simulation apparatus 100 is configured to display a plurality of landmarks estimated from the medical image of a user on the screen, and then display the A-space defined by the plurality of landmarks on the screen.

In addition, the simulation apparatus 100 is configured to display a cervical artificial disc model, which is a most suitable model for the A-space generated through S500, on a screen, S600. For example, a cervical artificial disc model modeled according to a height (e.g. distance between upper vertex of a cranial (upper) plate and lower vertex of a caudal (lower) plate), a length (e.g., width of a major axis), a width (e.g., width of a minor axis), a shape (surface or edge), or a combination thereof (e.g., a specification of a cervical artificial disc model) that is optimally suitable for the A-space is displayed on the screen.

In addition, the simulation apparatus 100 is configured to display a movement of bending or unbending a neck when the cervical artificial disc model displayed on the screen through S600 is applied to the surgical site, S700.

In addition, if a simulation performed by combining the cervical artificial disc model with the medical image of an actual user is completed, the simulation apparatus 100 is configured to finally determine a specification of the cervical artificial disc model to be applied to a user with reference to the simulation result and display the cervical artificial disc model modeled according to the determined specification on the screen, S800.

As described above, embodiments according to the present invention have been described with reference to the drawings, but these are merely examples, and it will be understood by a person skilled in the art that various modifications and other equivalent embodiments are possible therefrom. Therefore, the technical scope of the present invention should be determined by the following claims.

According to the present invention, since a process of generating an available space into which a cervical artificial disc of a surgery scheduled user is to be inserted by using a plurality of landmarks, and a process of simulating movement of the cervical artificial disc model designed to be optimized in the available space are intuitively and conveniently processed through a user interface, the cervical artificial disc can be manufactured in a customized manner with reference to the simulation result, the success probability of surgery of the cervical artificial disc replacement surgery is increased, and the satisfaction of a user related to the surgery prognosis is improved.

What is claimed is:

1. An apparatus for simulating medical procedure performing cervical artificial disc surgery, the apparatus comprising:
an available space display part configured to display an available space defined by a plurality of landmarks estimated from a medical image of a user;
a cervical artificial disc model display part configured to display the cervical artificial disc modeled to fit the available space; and
a simulation response display part configured to display a movement of bending or unfolding a neck when the cervical artificial disc is applied to a surgical site.

2. The apparatus of claim 1, further comprises:
a medical image display part configured to display the medical image of the user;
a surgical site area display part configured to display the surgical site area according to the designation of the surgical site area of an expert who has examined the medical image of the user; and
a landmark display part configured to display the plurality of landmarks estimated from the surgical site area.

3. The apparatus of claim 2, further comprises:
an A-space inspection part configured to inspect an A-space by comparing the A-space, which is an available space defined by the plurality of landmarks estimated from the medical image of the user, with a disc space of the surgery site;
a cervical artificial disc coupling part configured to apply a cervical artificial disc model modeled according to the A-space to the surgical site of the user to be operated; and
a simulation condition setting part configured to set a condition for simulating the movement of the neck when the cervical artificial disc model is applied to the surgical site of the user to be operated,
wherein the simulation result according to the set simulation condition is displayed through the simulation response display part.

4. The apparatus of claim 1, wherein the available space is the A-space defined by the plurality of landmarks, and the A-space is that a space between two unit cervical vertebrae to which the cervical artificial disc is implanted is modeled using the plurality of landmarks.

5. The apparatus of claim 1, wherein the plurality of landmarks are configured to be set:
at a center and left and right sides of a front outer region of a lower end of an upper end unit cervical vertebra to a direction of a skull, a cranial anterior center, a cranial anterior right, and a cranial anterior left, respectively;
at the center of the lower end of the upper end unit cervical vertebra to the direction of the skull, a cranial apex;
at a center and left and right sides of a rear outer region of the lower end of the upper end unit cervical vertebra to the direction of the skull, a cranial posterior center, a cranial posterior right, and a cranial posterior left, respectively;
at a center and left and right sides of a front outer region of an upper end of a lower end unit caudal vertebra to a direction of a spine, a caudal anterior center, a caudal anterior near right, a caudal anterior far right, a caudal anterior near left, and a caudal anterior far left, respectively; and
at a center and left and right sides of a rear outer region of the upper end of the lower end unit caudal vertebra to the direction of the spine, a caudal posterior center, a caudal posterior near right, a caudal posterior far right, a caudal posterior near left, and a caudal posterior far left, respectively.

6. A method for simulating medical procedure performing cervical artificial disc surgery in an apparatus thereof, the method comprising:
displaying an available space defined by a plurality of landmarks estimated from a medical image of a user;
displaying a cervical artificial disc modeled to fit the available space; and
displaying a movement of bending or unfolding a neck when the cervical artificial disc is applied to a surgical site.

7. The method of claim 6, further comprises:
displaying a medical image of a user;
displaying the surgical site area according to a designation of the surgical site area by an expert who has examined the medical image of the user; and
displaying the plurality of landmarks estimated from the surgical site area.

8. The method of claim 7, further comprises:
inspecting an A-space by comparing the A-space, which is an available space defined by the plurality of landmarks estimated from the medical image of the user, with a disc space of the surgery site;
applying a cervical artificial disc model modeled according to the A-space to a surgical site of the user to be operated; and
setting a condition for simulating the movement of the neck when the cervical artificial disc model is applied to a surgical site of the user to be operated,
wherein the simulation apparatus is configured to display the simulation result according to the set simulation condition on a screen.

9. The method of claim 6, wherein the available space is an A-space defined by the plurality of landmarks, and the A-space is that a space between two unit cervical vertebrae to which the cervical artificial disc is implanted is modeled using the plurality of landmarks.

10. The method of claim 6, wherein the plurality of landmarks are configured to be set:
at a center and left and right sides of a front outer region of a lower end of an upper end unit cervical vertebra to a direction of a skull, a cranial anterior center, a cranial anterior right, and a cranial anterior left, respectively;
at the center of the lower end of the upper end unit cervical vertebra to the direction of the skull, a cranial apex;
at a center and left and right sides of a rear outer region of the lower end of the upper end unit cervical vertebra to the direction of the skull, a cranial posterior center, a cranial posterior right, and a cranial posterior left, respectively;
at a center and left and right sides of a front outer region of an upper end of a lower end unit caudal vertebra to a direction of a spine, a caudal anterior center, a caudal anterior near right, a caudal anterior far right, a caudal anterior near left, and a caudal anterior far left, respectively; and
at a center and left and right sides of a rear outer region of the upper end of the lower end unit caudal vertebra to the direction of the spine, a caudal posterior center, a caudal posterior near right, a caudal posterior far right, a caudal posterior near left, and a caudal posterior far left, respectively.
